(19) United States
(12) Patent Application Publication
(10) Pub. No.: US 2024/0335234 A1
(43) Pub. Date: Oct. 10, 2024

(54) LITHOTRIPSY GUIDE SYSTEM

(71) Applicant: GYRUS ACMI, INC. D/B/A OLYMPUS SURGICAL TECHNOLOGIES AMERICA, Westborough, MA (US)

(72) Inventors: Joey Magno, Dudley, MA (US); Charles A. Baker, Rogers, MN (US)

(21) Appl. No.: 18/619,377

(22) Filed: Mar. 28, 2024

Related U.S. Application Data
(60) Provisional application No. 63/494,868, filed on Apr. 7, 2023.

(51) Int. Cl. A61B 18/26; A61B 17/00

(52) U.S. Cl. CPC A61B 18/26; A61B 2017/00296

(57) ABSTRACT

A laser lithotripsy guide system for guiding a laser fiber during a lithotripsy procedure may include a guide which may include a proximal end portion and a distal end portion. The guide may define a guide bore to receive a laser fiber therethrough. The guide may include at least one prong on the distal end portion. The guide system may also include a mounting assembly which may be securable to the laser fiber. The mounting assembly may be releasably insertable into the proximal end portion of the guide to releasably secure the mounting assembly to the guide.

### PRIORITY CLAIM

This application claims the benefit of priority to U.S. Provisional Patent Application Ser. No. 63/494,868, filed Apr. 7, 2023, the contents of which are hereby incorporated by reference in their entirety.

### TECHNOLOGICAL FIELD

The present disclosure relates generally to medical devices and instruments configured to provide diagnostic and treatment operations. More specifically, the present disclosure relates to a guide system that can be used during a lithotripsy procedure.

### BACKGROUND

A stone can form within organs of the human body such as within a kidney. In some cases, the stone is unable pass through the organs naturally and surgical intervention is required to remove the stone. In many cases, the stone must be broken into smaller pieces for extraction from a body lumen, such as a kidney or urinary tract. A lithotripsy device can be used to break and extract the stone. Common modalities of lithotripsy include laser lithotripsy, ultrasonic lithotripsy, and mechanical lithotripsy. In each of these modalities, energy can be delivered to the stone from the lithotripsy device to break the stone into smaller pieces for removal.

### SUMMARY

In an example a laser lithotripsy guide system for guiding a laser fiber during a lithotripsy procedure may include a guide which may include a proximal end portion and a distal end portion. The guide may define a guide bore to receive a laser fiber therethrough. The guide may include at least one prong on the distal end portion. The guide system may also include a mounting assembly which may be securable to the laser fiber. The mounting assembly may be releasably insertable into the proximal end portion of the guide to releasably secure the mounting assembly to the guide.

In an example a method of operating a laser lithotripsy guide system for guiding a laser fiber during a lithotripsy procedure using an endoscope may include coupling the laser fiber to a guide system, the guide system including a proximal end portion and a distal end portion. The distal end portion of the guide system may include at least one prong, where an axial movement of the laser fiber is limited relative to the guide system. The laser fiber may be coupled such that a distal end of the laser fiber is a specified distance from a distal end of the at least one prong. The method may also include engaging a target with at least one of the at least one prong to provide a specified spacing between a tip of the laser fiber and the target and applying laser energy to the target.

In an example a laser lithotripsy guide system for guiding a laser fiber during a lithotripsy procedure may include a mounting assembly securable to a laser fiber. The guide system may also include a guide including a body configured to receive the mounting assembly at least partially therein to releasably secure the mounting assembly and the laser fiber to the guide, the body defining a guide bore to receive the laser fiber therethrough when the mounting assembly is secured to the guide. The guide may also include at least one prong extending distally from the body, the at least one prong configured to at least partially surround the laser fiber when the laser fiber extends through the guide bore.

### BRIEF DESCRIPTION OF THE DRAWINGS

In the drawings, which may not be drawn to scale, like numerals may describe substantially similar components throughout one or more of the views. Like numerals having different letter suffixes may represent different instances of substantially similar components. The drawings illustrate generally, by way of example but not by way of limitation.

FIG. 1 is a cross sectional view of an example of portions of a lithotripsy guide system and portions of an environment in which the guide system may be used.

FIG. 2 is an exploded perspective view of an example of portions of a mounting assembly of the lithotripsy guide system of FIG. 1.

FIG. 3 is a perspective view of an example of portions of a guide of the lithotripsy guide system of FIG. 1.

FIG. 4 is an end view of an example of portions of the lithotripsy guide system of FIG. 1.

FIG. 5 is a cross sectional view of an example of portions of the lithotripsy guide system of FIG. 1 in operation, including portions of an environment in which the guide system may be used.

FIG. 6 is a cross sectional view of an example of portions of the lithotripsy guide system of claim 1 showing the collet chuck and lock nut of the mounting assembly and the connection between the mounting assembly and guide.

FIG. 7 is a diagram showing an example of a method for operating portions of a lithotripsy guide system.

FIG. 8 is a block diagram of an example of portions of a machine upon which one or more portions of the present disclosure may be implemented.

### DETAILED DESCRIPTION

In one or more forms of lithotripsy, a specified spacing distance between the lithotripsy device and the stone may be desired. For example, in a laser lithotripsy procedure, it may be desirable to have the stone close enough to the laser fiber that the laser energy is delivered effectively, but far enough from the laser fiber that the laser fiber is not damaged during lithotripsy. Achieving the specified spacing distance may be complicated by the movement of the stone, such as may be due to retropulsion. Achieving the specified spacing distance may alternatively or additionally be complicated due to the difficulty in determining the distance between the lithotripsy device and stone during the procedure, such as by the operator's estimation or a lithotripsy system's calculation.

This disclosure provides solutions to the problem of achieving a specified spacing distance. That is, a desired specified spacing distance between the stone and the lithotripsy device can be achieved by using a guide system. This can help increase the amount of time that the stone is a specified spacing from the lithotripsy device, and save time during a lithotripsy procedure due to one or more of more efficient energy application or reduced time lost due to burned laser fibers.

In some examples, a mechanical guide system coupled to the laser fiber can be used to help achieve a desired spacing between the laser fiber and the stone. In some examples, a guide device with at least one prong can be used with the prong resting against the stone. In some examples, a suction pressure can be used to hold the stone against the guide.

The above discussion is intended to provide an overview of subject matter of the present patent application. It is not intended to provide an exclusive or exhaustive explanation of the invention. The description below is included to provide further information about the present patent application.

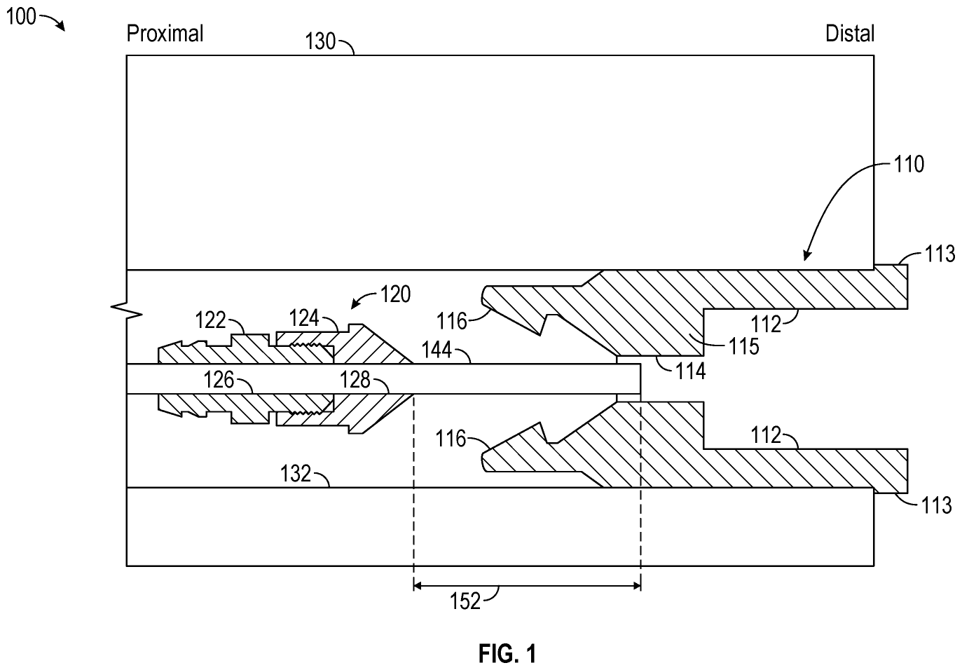

FIG. 1 is a cross sectional view of an example of portions of a lithotripsy guide system 100 and portions of an environment in which the guide system may be used. FIG. 1 shows that the guide system 100 may include a guide 110 and a mounting assembly 120. The environment in which the guide system 100 is used may include an endoscope 130.

The guide 110 may include at least one prong 112 on the distal end portion, and a body 115 coupled to the at least one prong 112. The guide 110 can also include a plurality of projections 116 on a proximal end portion of the body 115. The body 115 may at least partially define a bore 114 for receiving a laser fiber 144.

The guide 110 may include projections 113 connected to a distal portion of each prong 112. The projections 113 can extend radially outward or laterally outward from a distal portion of each prong 112. The projections 113 can be engageable with a distal portion of the endoscope 130 when the proximal end of the guide is inserted into the working channel 132. For example, an outer diameter of the guide 110 may be similar to an inner diameter of the working channel 132 such that the guide 110 may form a friction fit with the working channel 132.

The at least one prong 112 may include one prong, two prongs, three prongs, four prongs, five prongs, or six or more prongs. The at least one prong 112 may extend distally from a distal end of the body 115. The plurality of projections 116 may include two projections, three projections, four projections, five projections, or six or more projections. In an example, the projections 116 may be replaced by a continuous or substantially continuous projection with a similar radial shape to the projections 116. The projections 116 may extend proximally from a proximal end of the body 115. The prongs 112 may be evenly spaced circumferentially about an axis of the guide 110, such as may include being evenly spaced in a generally circular fashion. In an example, the guide 110 may include 3 prongs 112 spaced by 120 degrees.

The mounting assembly 120 may include a collet chuck 122 and a lock nut 124. The collet chuck 122 may define a collet chuck bore 126, which may receive the laser fiber 144 therethrough. The lock nut 124 may define a lock nut bore 128, which may align with the collet chuck bore 126 when the lock nut 124 is secured to the collet chuck 122 to receive the laser fiber 144 therethrough.

The collet chuck 122 may threadably engage with the lock nut 124, such as may cause the collet chuck 122 to clamp onto the laser fiber 144 by reducing a diameter of the collet chuck bore 126 and applying a clamping force to the laser fiber 144. The mounting assembly 120 may be coupled to the laser fiber 144 such that there is a calibrated distance 152 from the tip of the mounting assembly 120 (e.g., the tip of the lock nut 124) to the tip of the laser fiber 144.

The mounting assembly 120 may be releasably insertable into the guide 110. For example, the mounting assembly 120 may be shaped to mechanically couple with the guide 110 when a distal end portion of the mounting assembly 120 is inserted into a proximal end portion of the guide 110 between the projections 116. When the mounting assembly 120 is inserted into the projections 116, the guide system 100 may function and behave as if it is a single component as opposed to a combination of multiple components. The mounting assembly 120 may be released from the guide 110 when the mounting assembly 120 is pulled away from the guide 110.

The guide system 100 may be manufactured from a variety of materials, such as may include one or more of metals, plastics, or composites. The components of the guide system 100 may each be manufactured from the same material, or one or more of the components may be manufactured from a different type of material. In an example, one or more components of the guide system 100 may be manufactured from one or more of polyvinyl chloride (PVC), polypropylene (PP), polyethylene (PE), nylon, acrylonitrile butadiene styrene (ABS), or other thermoplastic. In an example, one or more of the guide 110, the collet chuck 122, or the lock nut 124 may be manufactured through one or more of injection molding (e.g., injection micro molding), an additive manufacturing process (e.g., 3D printing), or a subtractive machining process. In an example, the dimensions of the guide system 100 may be between 0.5 mm and 2 mm in diameter, and between 2 mm and 10 mm in axial length when the mounting assembly 120 is connected to the guide 110.

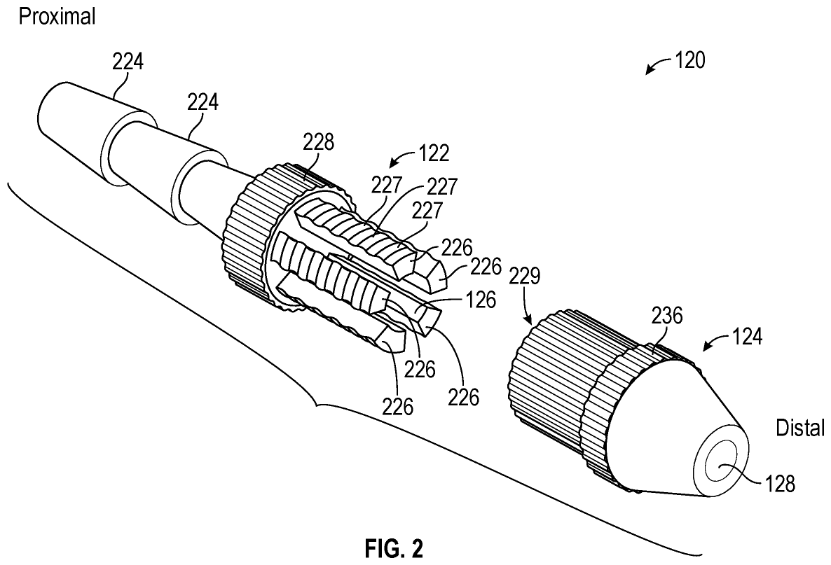

FIG. 2 is an exploded perspective view of an example of portions of a mounting assembly 120 of the lithotripsy guide system 100 of FIG. 1. FIG. 2 shows the collet chuck 122 in more detail, such as including a plurality of collet segments 226 on a distal end portion and at least one barb 224 on a proximal end portion. The plurality of collet segments 226 may include threads 227 on their outer surface to threadably engage with corresponding threads 229 that may be on an inner surface of the lock nut 124. The collet chuck 122 may include a textured outer portion 228 to allow a user to grip the collet chuck 122, such as may be helpful during one or more of threadably engaging the lock nut 124 to clamp the laser fiber 144 or threadably disengaging the lock nut 124 to release the laser fiber 144. Similarly, the lock nut 124 may include a textured outer portion 236 to allow a user to grip the lock nut 124, such as may be helpful during one or more of threadably engaging the collet chuck 122 to clamp the laser fiber 144 or threadably disengaging the collet chuck 122 to release the laser fiber 144.

One or more of the outer threads 227 of the collet chuck 122 or the inner threads 229 of the lock nut 124 may be tapered such that the collet segments 226 are constricted inward to clamp onto the laser fiber 144 as the lock nut 124 is threaded onto the collet chuck 122. In an example, the inner threads on the lock nut 124 may have a larger diameter at the proximal end than at the distal end. In an example, the outer threads on the collet chuck 122 may have a larger diameter at the proximal end than at the distal end. The collet chuck 122 and the lock nut 124 may be shaped to clamp onto a laser fiber with a specified diameter, or may be shaped to clamp onto a range of laser fiber diameters. The collet chuck 122 and the lock nut 124 may apply a predetermined clamping motion with a given number of turns of the lock nut 124 corresponding to a given constriction of the collet segments 226, such as may include one or more of a linear or nonlinear clamping motion.

The at least one barb 224 may be shaped to allow a substantially cylindrical tube to slot over the at least one barb 224 with a resistance that is less than the resistance required to pull the substantially circular tube off of the at least one barb 224. This may result in the at least one barb 224 securing the substantially cylindrical tube to the collet chuck 122. In an example, the at least one barb 224 may not be included on the collet chuck 122.

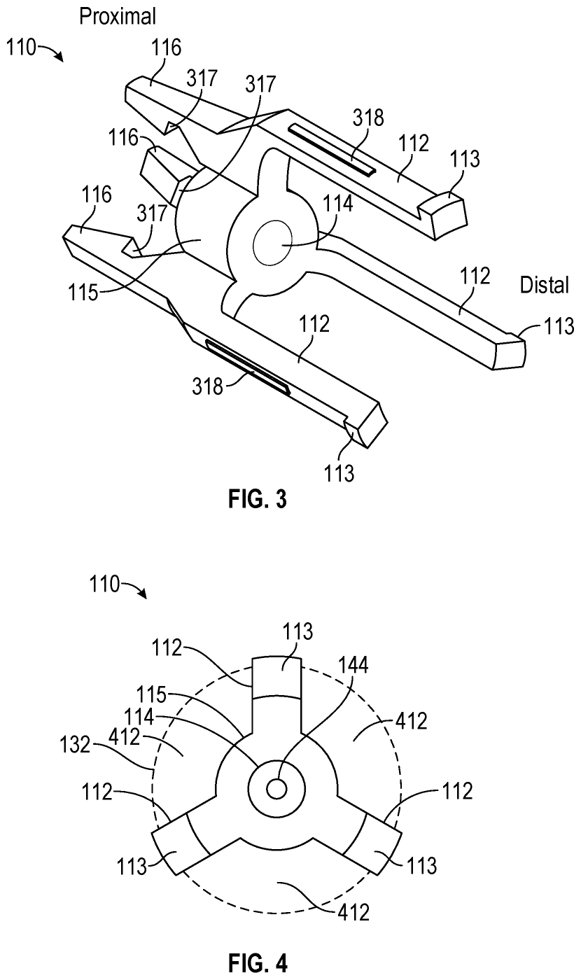

FIG. 3 is a perspective view of an example of portions of a guide 110 of the lithotripsy guide system of FIG. 1. FIG. 3 shows further details of the projections 113, which can be located on a laterally outer portion of the at least one prong 112. The projections 113 may be shaped to engage a distal end of the endoscope 130 to limit proximal movement of the guide 110 with respect to the endoscope 130. For example, the projection 113 may catch on the distal edge of the working channel 132 to help prevent the guide 110 from being pulled fully within the endoscope 130.

Each of the at least one prongs 112 may include a raised rib 318 on a radially outer portion of respective ones of the at least one prong 112. The raised rib 318 may engage the inside of the working channel 132 and may increase friction between the guide 110 and the working channel 132 to help maintain a position or orientation of the guide 110 with respect to the working channel 132.

The raised rib 318 may be manufactured from the same material as the rest of the guide 110, or the raised rib 318 may be manufactured from a different material than the rest of the guide 110. In an example, the raised rib 318 may be manufactured from a material with a higher coefficient of friction than the remainder of the guide 110. In an example, a chemical may be applied to the raised rib 318 to increase friction. In an example, the raised rib 318 may be mechanically altered to increase friction, such as may include roughing a surface of the raised rib 318.

One or more of the plurality of projections 116 may include a retaining clip 317 on a radially inner side. Together, the plurality of projections 116 may define a receiving interface to accept the mounting assembly 120. The retaining clip 317 may be shaped to releasably secure the mounting assembly 120 to the guide 110 when the proximal end portion of the mounting assembly 120 is inserted into the distal end portion of the guide 110. The plurality of projections 116 may be configured to mechanically reversibly deflect (such as elastically, e.g., without deformation of the projections 116 or the guide 110) radially outward as the mounting assembly 120 is inserted into the guide 110, and then deflect back radially inward when the mounting assembly 120 clears the retaining clip 317.

One or more of the plurality of projections 116 may be axially aligned with one or more of the prongs 112. This may result in the projection 116 and the prong 112 forming a continuous section that is joined with the body 115 in a single location. In an example, there may be three projections 116 that are axially aligned with respective ones of three prongs 112. In an example, there may be more prongs 112 than projections 116. In an example, there may be less prongs 112 than projections 116. For example, using less prongs 112 than projections 116 may allow for better visual access or less resistance to fluid flow, such as may be due to the prongs 112 having a larger diameter than the projections 116.

FIG. 4 is an end view of an example of portions of the lithotripsy guide system 100 of FIG. 1. FIG. 4 shows an end view of the guide 110 from a proximal perspective. FIG. 4 shows that the prongs 112 may define at least one pathway 412 axially through the guide 110 and guide system 100 for fluid flow or visual access. For example, a camera may be positioned to view through at least one pathway 412 between two of the prongs 112. In an example, there may be a single prong 112, and the radial area not covered by the single prong 112 may define the at least one pathway 412.

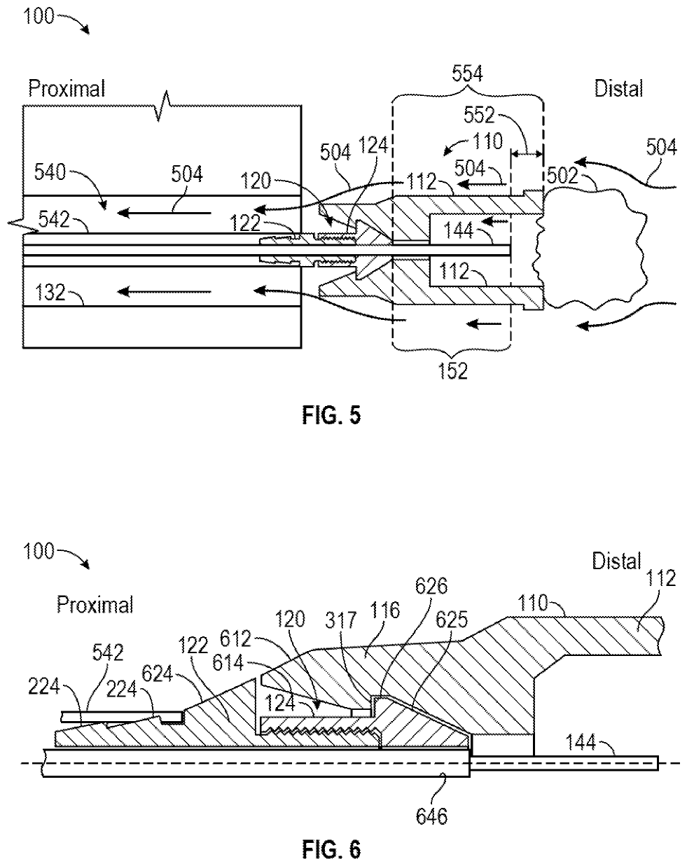

FIG. 5 is a cross sectional view of an example of portions of the lithotripsy guide system 100 of FIG. 1 in operation, including portions of an environment in which the guide system may be used. FIG. 5 shows the mounting assembly 120 coupled with the guide 110. FIG. 5 shows that the environment may include a target 502, such as may include a kidney or ureteral stone. The tips of the prongs 112 may engage a surface of the target 502. This may create a desired spacing 552 between the tip of the laser fiber 144 and the surface of the target 502. For example, the desired spacing 552 may be created by adjusting the calibrated distance 152 based upon a specified total distance 554 between the tip of the at least one prong 112 and the tip of the mounting assembly 120 when the mounting assembly 120 is inserted into the guide 110. The calibrated distance 152 necessary to achieve the desired spacing 552 may be calculated according to equation 1.

D₁=D₂−D₃ Equation 1

In equation 1, D₁ is the calibrated distance 152, D₂ is the specified total distance 554, and D₃ is the desired spacing 552. In an example, the desired spacing may be 0.5 mm, 1 mm, 1.5 mm, 2 mm, 2.5 mm, or 3 mm. The desired spacing may be selected to help optimize an efficiency with which lithotripsy energy is applied to a stone, balanced with the adverse effects of close spacing on one or more portions of the lithotripsy device.

FIG. 5 also shows fluid flow 504 due to suction applied by the endoscope 130. The fluid flow 504 may travel around the target 502, around and/or through the guide system 100, such as may include through the at least one pathway 412, and through the working channel 132. The fluid flow 504 may result in a force on the target 502 that acts to hold the target 502 against the prongs 112. This suction force may minimize the effects of retropulsion where a stone is pushed away by the laser energy. This may help maintain the desired spacing 552 between the tip of the laser fiber 144 and the target 502, such as may be due to cancelling out retropulsion of the target 502 due to the lithotripsy energy. The fluid flow 504 may be provided by one or more of an irrigation or suction system of the endoscope 130.

The laser fiber 144 may be a part of a laser fiber assembly 540. The laser fiber assembly 540 may also include an outer sheath 542, such as may include a column sheath. The outer sheath 542 may stiffen the laser fiber assembly 540, such as to increase one or more of an axial compression or axial tension strength of the laser fiber assembly 540. For example, the laser fiber 144 alone may not have sufficient axial strength to couple the lock nut 124 with the guide 110 or decouple the lock nut 124 from the guide 110.

FIG. 6 is a cross sectional view of an example of portions of the lithotripsy guide system 100 of claim 1 showing the collet chuck 122 and lock nut 124 of the mounting assembly 120 and the connection between the mounting assembly 120 and guide 110. FIG. 6 shows that the laser fiber 144 may be surrounded by cladding 646. The cladding 646 may help provide rigidity to the laser fiber 144 or protect the laser fiber 144 from mechanical damage. Alternatively or in addition, the cladding 646 may help limit external light from entering the laser fiber 144. The cladding 646 may extend along a length of the laser fiber 144. The cladding 646 may be stripped back from the tip of the laser fiber 144, such as may include stripping flush with the distal end of the lock nut 124, as shown in FIG. 6.

In addition to the cladding 646, there may be an outer sheath 542 over the laser fiber 144 and the cladding 646. The outer sheath 542 may provide one or more of additional structural rigidity or mechanical protection. The outer sheath 542 may engage with the at least one barb 224 on the collet chuck 122 to couple with the collet chuck 122. The outer sheath 542 may provide additional rigidity to support or stabilize the mounting assembly 120 when the mounting assembly 120 is being coupled with the guide 110. Alternatively or additionally, the outer sheath 542 may provide additional rigidity to the guide system 100 when the guide system 100 is being used in a lithotripsy procedure.

The projections 116 may include a radially inner chamfer 614 that may help direct the lock nut 124 into the receiving interface, such as may include axially aligning the lock nut 124 with the receiving interface of the guide 110. The lock nut 124 may include a radially outer chamfer 625 that may help direct the lock nut 124 into the receiving interface.

The projections 116 may include a radially outer chamfer 612 that may help direct the guide system 100 or the guide 110 into the working channel 132. The collet chuck 122 may include a radially outer chamfer 624 that may help direct the guide system 100 or the guide 110 into the working channel 132. In an example, the radially outer chamfer 612 and the radially outer chamfer 624 may substantially align to provide one substantially continuous radially outer chamfer to help axially align the guide system 100 with the working channel 132 for or during insertion of the guide 110 into the working channel 132.

The plurality of projections 116 may reversibly deflect as the lock nut enters the receiving interface. The projections 116 may return to an original position when the mechanical snap ridge 626 clears the retaining clip 317, which may result in the lock nut 124 being mechanically coupled to the guide 110. The lock nut 124 may include a mechanical snap ridge 626 at least partially defining a portion of the lock nut 124 with a larger radius or diameter. The mechanical snap ridge 626 can at least partially define an axial surface that may engage with at least a portion of the retaining clip 317 to limit movement of the lock nut 124 with respect to the guide 110, such as to mechanically hold the lock nut 124 and guide 110 together.

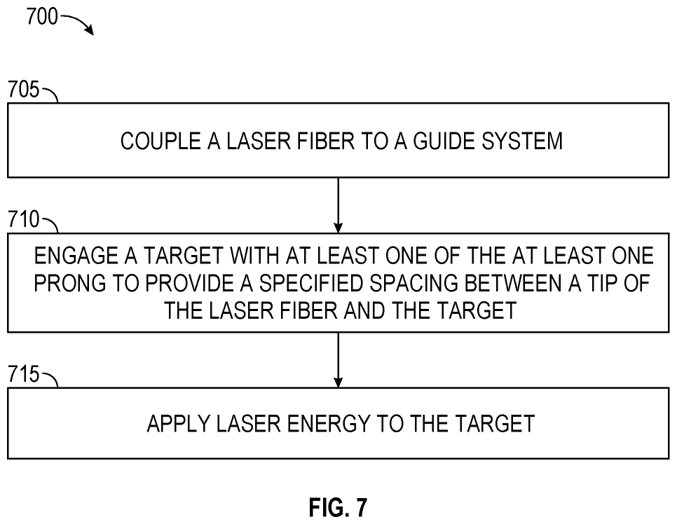

FIG. 7 is a diagram showing an example of a method 700 for operating portions of a lithotripsy guide system 100. At 705, a guide system can be coupled to a laser fiber. The guide system may include a proximal end portion and a distal end portion. The distal end portion of the guide system may include at least one prong. An axial movement of the laser fiber may be limited relative to the guide system. The laser fiber may be coupled such that a distal end of the laser fiber is a specified distance from a distal end of the at least one prong. At 710, a target can be engaged with at least one of the at least one prong to provide a specified spacing between a tip of the laser fiber and the target. At 715, laser energy can be applied to the target. The shown order of steps is not intended to be a limitation on the order the steps are performed in. In an example, two or more steps may be performed simultaneously or at least partially concurrently.

The guide system coupled to the laser fiber at step 705 can include a guide system 100 including a guide 110 and a mounting assembly 120. The guide 110 may be inserted into a distal end of an endoscope 130 working channel 132, such as may include inserting the guide 110 into the working channel 132 before the endoscope 130 is inserted into a patient to perform a procedure. The laser fiber 144 may be coupled to the guide mounting assembly 120 externally to the patient and the endoscope 130, and then the laser fiber 144 and mounting assembly 120 may be passed through the working channel 132 while coupled. During the external laser fiber 144 coupling process, an axial position of the laser fiber 144 within the mounting assembly 120 may be set or adjusted such that there is a desired spacing 552 between the tip of the laser fiber 144 and the target 502 when the mounting assembly 120 is coupled with the guide 110.

Once the mounting assembly 120 coupled to the laser fiber 144 has been passed through the working channel 132 and nears a distal end of the working channel 132, the lock nut 124 may enter the receiving interface of the guide 110. In an example, the mounting assembly 120 coupled to the laser fiber 144 may be passed through the working channel 132 after the endoscope 130 is inserted into the patient. In an example, the mounting assembly 120 coupled to the laser fiber 144 may be passed through the working channel 132 before the endoscope 130 is inserted into the patient. The lock nut 124 may be guided into the receiving interface with the help of one or more chamfers, such as may include radially inner chamfer 614 or radially outer chamfer 625. A force applied to the mounting assembly 120 through the laser fiber 144 (including the cladding 646 and outer sheath 542 as may be applicable) may reversibly deflect the plurality of projections 116 and releasably couple the mounting assembly 120 with the guide 110.

Following the coupling of the mounting assembly 120 with the guide 110, a force applied to the guide system 100 through the laser fiber 144 (including the cladding 646 and outer sheath 542 as may be applicable) may push the guide system 100 at least partially outside the working channel 132 to engage with the target 502 at step 710. The mounting assembly 120 and guide 110 may be configured such that a first force to couple the mounting assembly 120 with the guide 110 is less than a second force required to push the guide 110 out of the endoscope 130. This may help ensure that the mounting assembly 120 and guide 110 are coupled before the guide 110 is pushed out of the endoscope 130. If the guide 110 is pushed out of the endoscope 130 before coupling with the mounting assembly 120, it may be difficult to couple the guide 110 with the mounting assembly 120. An axial pushing strength of the laser fiber 144 (including the cladding 646 and outer sheath 542 as may be applicable) may exceed both the first and second force.

Suction may be applied during step 715, such as may help to hold the target against the at least one prong 112 and counteract the retropulsion caused by the lithotripsy energy. Alternatively or additionally during step 715, a lateral friction force between the at least one prong 112 and the target 502 may hold the target 502 in place laterally to help with one or more of positioning or aiming of the laser fiber. This may help to counteract lateral forces do to one or more of fluid flow, forces from external tissue, or lithotripsy energy.

Once a desired level of laser energy has been applied to the target 502 at step 715, or the laser fiber 144 needs tending, the guide system 100 may be retracted into the working channel 132 by pulling on the laser fiber 144 (including the cladding 646 and outer sheath 542 as may be applicable). The guide system 100, including the guide 110 and mounting assembly 120, may be axially aligned with the working channel 132 with the help of one or more chamfers, such as may include radially outer chamfer 612 or radially outer chamfer 624. After the guide system 100 is retracted into the working channel 132, the mounting assembly 120 may be decoupled, from the guide 110 by pulling on the laser fiber 144 (including the cladding 646 and outer sheath 542 as may be applicable). The mounting assembly 120 and guide 110 may be configured such that a third force required to seat the guide 110 in the working channel 132 may be less than a fourth force required to decouple the mounting assembly 120 from the guide 110. This may help ensure that the guide 110 is seated in the working channel 132 before the mounting assembly 120 is decoupled from the guide 110. For example, once the mounting assembly 120 is decoupled from the guide 110, there may not be a way to apply a force to the guide 110 to provide a desired coupling with the working channel 132. An axial pulling strength of the laser fiber 144 (including the cladding 646 and outer sheath 542 as may be applicable) may exceed both the third and fourth force.

Once the mounting assembly 120 is decoupled from the guide 110, the mounting assembly 120 and laser fiber 144 can be removed from the working channel 132. The procedure may be ended, or the laser fiber 144 may be replaced, trimmed, or adjusted, and the procedure may continue.

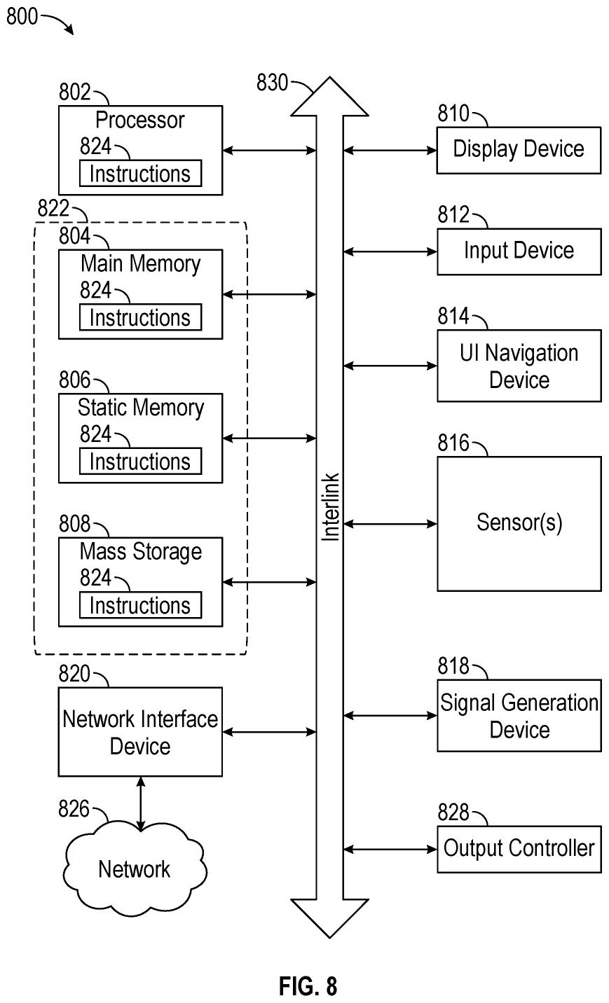

FIG. 8 is a block diagram of an example of portions of a machine 800 upon which one or more portions of the present disclosure may be implemented. Examples, as described herein, may include, or may operate by, logic or a number of components, or mechanisms in the machine 800. Circuitry (e.g., processing circuitry) is a collection of circuits implemented in tangible entities of the machine 800 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 800 follow.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 800 may include a hardware processor 802 (e.g., a central processing unit (CPU), a graphics processing unit (GPU), a hardware processor core, or any combination thereof), a main memory 804, a static memory (e.g., memory or storage for firmware, microcode, a basic-input-output (BIOS), unified extensible firmware interface (UEFI), etc.) 806, and mass storage 808 (e.g., hard drives, tape drives, flash storage, or other block devices) some or all of which may communicate with each other via an interlink (e.g., bus) 830. The machine 800 may further include a display unit 810, an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 808, a signal generation device 818 (e.g., a speaker), a network interface device 820, and one or more sensors 816, such as a global positioning system (GPS) sensor, compass, accelerometer, or other sensor. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 802, the main memory 804, the static memory 806, or the mass storage 808 may be, or include, a machine readable medium 822 on which is stored one or more sets of data structures or instructions 824 (e.g., software) embodying or utilized by any one or more of the techniques or functions described herein. The instructions 824 may also reside, completely or at least partially, within any of registers of the processor 802, the main memory 804, the static memory 806, or the mass storage 808 during execution thereof by the machine 800. In an example, one or any combination of the hardware processor 802, the main memory 804, the static memory 806, or the mass storage 808 may constitute the machine readable media 822. While the machine readable medium 822 is illustrated as a single medium, the term “machine readable medium” may include a single medium or multiple media (e.g., a centralized or distributed database, and/or associated caches and servers) configured to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine readable medium 822 may be representative of the instructions 824, such as instructions 824 themselves or a format from which the instructions 824 may be derived. This format from which the instructions 824 may be derived may include source code, encoded instructions (e.g., in compressed or encrypted form), packaged instructions (e.g., split into multiple packages), or the like. The information representative of the instructions 824 in the machine readable medium 822 may be processed by processing circuitry into the instructions to implement any of the operations discussed herein. For example, deriving the instructions 824 from the information (e.g., processing by the processing circuitry) may include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 824.

In an example, the derivation of the instructions 824 may include assembly, compilation, or interpretation of the information (e.g., by the processing circuitry) to create the instructions 824 from some intermediate or preprocessed format provided by the machine readable medium 822. The information, when provided in multiple parts, may be combined, unpacked, and modified to create the instructions 824. For example, the information may be in multiple compressed source code packages (or object code, or binary executable code, etc.) on one or several remote servers. The source code packages may be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 824 may be further transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 802.11 family of standards known as Wi-Fi®, IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software. A transmission medium is a machine readable medium.

### ADDITIONAL NOTES & EXAMPLES

Example 1 is a laser lithotripsy guide system for guiding a laser fiber during a lithotripsy procedure, the guide system comprising: a guide including a proximal end portion and a distal end portion, the guide defining a guide bore to receive a laser fiber therethrough, the guide including at least one prong on the distal end portion; and a mounting assembly securable to the laser fiber, the mounting assembly releasably insertable into the proximal end portion of the guide to releasably secure the mounting assembly to the guide.
In Example 2, the subject matter of Example 1 optionally includes wherein the at least one prong defines one or more pathways to provide at least one of fluid flow or visual access through the guide.
In Example 3, the subject matter of any one or more of Examples 1-2 optionally include wherein the at least one prong is engageable with a distal portion of a working channel of an endoscope to releasably secure the guide to the endoscope when the proximal end portion of the guide is inserted into the working channel.
In Example 4, the subject matter of Example 3 optionally includes wherein one or more of the at least one prong include a projection located on a laterally outer portion of the one or more of the at least one prong to engage the working channel and limit proximal movement of the guide with respect to the working channel.
In Example 5, the subject matter of any one or more of Examples 1-4 optionally include wherein the mounting assembly includes barbs on a proximal end portion of the mounting assembly to secure the mounting assembly to a column sheath of the laser fiber.
In Example 6, the subject matter of any one or more of Examples 1-5 optionally include wherein the mounting assembly includes: a collet chuck defining a collet chuck bore to receive the laser through the collet chuck; and a lock nut defining a lock nut bore to receive the laser fiber through the lock nut, the lock nut threadably securable to the collet chuck to cause the collet chuck to clamp the laser fiber.
In Example 7, the subject matter of Example 6 optionally includes wherein the guide includes a receiving interface defined by a plurality of projections extending from the proximal end portion of the guide, the plurality of projections configured to reversibly deflect as the lock nut, engageable with the plurality of projections, enters the receiving interface.
In Example 8, the subject matter of Example 7 optionally includes wherein a first force required to releasably insert the lock nut into the receiving interface is less than a second force required to decouple the guide from a working channel of an endoscope.
In Example 9, the subject matter of any one or more of Examples 6-8 optionally include wherein at least one of the collet chuck or the proximal end portion of the guide is chamfered on a radially outer side to axially align the guide and a working channel of an endoscope during coupling of the guide with the working channel.
In Example 10, the subject matter of any one or more of Examples 6-9 optionally include wherein the proximal end portion of the guide is chamfered on a radially inner side to axially align the guide and the mounting assembly during coupling of the guide and the mounting assembly.
In Example 11, the subject matter of any one or more of Examples 1-10 optionally include prongs.
Example 12 is a method of operating a laser lithotripsy guide system for guiding a laser fiber during a lithotripsy procedure using an endoscope, the method comprising: coupling the laser fiber to a guide system, the guide system including a proximal end portion and a distal end portion, the distal end portion of the guide system including at least one prong, wherein an axial movement of the laser fiber is limited relative to the guide system, wherein the laser fiber is coupled such that a distal end of the laser fiber is a specified distance from a distal end of the at least one prong; engaging a target with at least one of the at least one prong to provide a specified spacing between a tip of the laser fiber and the target; and applying laser energy to the target.
In Example 13, the subject matter of Example 12 optionally includes applying a suction force to hold the target against at least one of the at least one prong.
In Example 14, the subject matter of any one or more of Examples 12-13 optionally include inserting a distal end portion of a mounting assembly, the mounting assembly coupled to the laser fiber, into a proximal end portion of a guide, the guide including the at least one prong, to removably couple the guide to the mounting assembly and to secure the laser fiber to the guide, the mounting assembly and guide comprising the guide system.
In Example 15, the subject matter of Example 14 optionally includes inserting the guide into a distal end of a working channel of the endoscope before inserting the endoscope into a patient, the guide engaging the working channel of the endoscope.
In Example 16, the subject matter of Example 15 optionally includes passing the mounting assembly, coupled to the laser fiber, from a proximal end of the endoscope working channel through the working channel to removably couple with the guide on the distal end of the working channel.
In Example 17, the subject matter of Example 16 optionally includes wherein the passing the laser fiber mounting assembly occurs after inserting the endoscope into the patient.
In Example 18, the subject matter of any one or more of Examples 16-17 optionally include before the passing the laser fiber mounting assembly, adjusting an axial position of the laser fiber within the laser fiber mounting assembly such that when the laser fiber mounting assembly is coupled with the guide, there is the specified spacing between the tip of the laser fiber and a distal end of the at least one prong.
Example 19 is a laser lithotripsy guide system for guiding a laser fiber during a lithotripsy procedure, the guide system comprising: a mounting assembly securable to a laser fiber; and a guide comprising: a body configured to receive the mounting assembly at least partially therein to releasably secure the mounting assembly and the laser fiber to the guide, the body defining a guide bore to receive the laser fiber therethrough when the mounting assembly is secured to the guide; and at least one prong extending distally from the body, the at least one prong configured to at least partially surround the laser fiber when the laser fiber extends through the guide bore.
In Example 20, the subject matter of Example 19 optionally includes wherein the mounting assembly includes: a collet chuck defining a collet chuck bore to receive the laser through the collet chuck; and a lock nut defining a lock nut bore to receive the laser fiber through the lock nut, the lock nut threadably securable to the collet chuck to cause the collet chuck to clamp the laser fiber.
Example 21 is at least one machine-readable medium including instructions that, when executed by processing circuitry, cause the processing circuitry to perform operations to implement of any of Examples 1-20.
Example 22 is an apparatus comprising means to implement of any of Examples 1-20.
Example 23 is a system to implement of any of Examples 1-20.
Example 24 is a method to implement of any of Examples 1-20.

Each of the non-limiting aspects above can stand on its own or can be combined in various permutations or combinations with one or more of the other aspects or other subject matter described in this document.

The above detailed description includes references to the accompanying drawings, which form a part of the detailed description. The drawings show, by way of illustration, specific embodiments in which the invention can be practiced. These embodiments are also referred to generally as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc., are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Such instructions can be read and executed by one or more processors to enable performance of operations comprising a method, for example. The instructions are in any suitable form, such as but not limited to source code, compiled code, interpreted code, executable code, static code, dynamic code, and the like.

Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A laser lithotripsy guide system for guiding a laser fiber during a lithotripsy procedure, the guide system comprising:
a guide including a proximal end portion and a distal end portion, the guide defining a guide bore to receive a laser fiber therethrough, the guide including at least one prong on the distal end portion; and
a mounting assembly securable to the laser fiber, the mounting assembly releasably insertable into the proximal end portion of the guide to releasably secure the mounting assembly to the guide.

2. The guide system of claim 1, wherein the at least one prong defines one or more pathways to provide at least one of fluid flow or visual access through the guide.

3. The guide system of claim 1, wherein the at least one prong is engageable with a distal portion of a working channel of an endoscope to releasably secure the guide to the endoscope when the proximal end portion of the guide is inserted into the working channel.

4. The guide system of claim 3, wherein one or more of the at least one prong include a projection located on a laterally outer portion of the one or more of the at least one prong to engage the working channel and limit proximal movement of the guide with respect to the working channel.

5. The guide system of claim 1, wherein the mounting assembly includes barbs on a proximal end portion of the mounting assembly to secure the mounting assembly to a column sheath of the laser fiber.

6. The guide system of claim 1, wherein the mounting assembly includes:
a collet chuck defining a collet chuck bore to receive the laser through the collet chuck; and
a lock nut defining a lock nut bore to receive the laser fiber through the lock nut, the lock nut threadably securable to the collet chuck to cause the collet chuck to clamp the laser fiber.

7. The guide system of claim 6, wherein the guide includes a receiving interface defined by a plurality of projections extending from the proximal end portion of the guide, the plurality of projections configured to reversibly deflect as the lock nut, engageable with the plurality of projections, enters the receiving interface.

8. The guide system of claim 7, wherein a first force required to releasably insert the lock nut into the receiving interface is less than a second force required to decouple the guide from a working channel of an endoscope.

9. The guide system of claim 6, wherein at least one of the collet chuck or the proximal end portion of the guide is chamfered on a radially outer side to axially align the guide and a working channel of an endoscope during coupling of the guide with the working channel.

10. The guide system of claim 6, wherein the proximal end portion of the guide is chamfered on a radially inner side to axially align the guide and the mounting assembly during coupling of the guide and the mounting assembly.

11. The guide system of claim 1, wherein the at least one prong includes 3 prongs.

12. A method of operating a laser lithotripsy guide system for guiding a laser fiber during a lithotripsy procedure using an endoscope, the method comprising:
coupling the laser fiber to a guide system, the guide system including a proximal end portion and a distal end portion, the distal end portion of the guide system including at least one prong, wherein an axial movement of the laser fiber is limited relative to the guide system, wherein the laser fiber is coupled such that a distal end of the laser fiber is a specified distance from a distal end of the at least one prong;
engaging a target with at least one of the at least one prong to provide a specified spacing between a tip of the laser fiber and the target; and
applying laser energy to the target.

13. The method of claim 12, further comprising applying a suction force to hold the target against at least one of the at least one prong.

14. The method of claim 12, further comprising:
inserting a distal end portion of a mounting assembly, the mounting assembly coupled to the laser fiber, into a proximal end portion of a guide, the guide including the at least one prong, to removably couple the guide to the mounting assembly and to secure the laser fiber to the guide, the mounting assembly and guide comprising the guide system.

15. The method of claim 14, further comprising:
inserting the guide into a distal end of a working channel of the endoscope before inserting the endoscope into a patient, the guide engaging the working channel of the endoscope.

16. The method of claim 15, further comprising:
passing the mounting assembly, coupled to the laser fiber, from a proximal end of the endoscope working channel through the working channel to removably couple with the guide on the distal end of the working channel.

17. The method of claim 16, wherein the passing the laser fiber mounting assembly occurs after inserting the endoscope into the patient.

18. The method of claim 16, further comprising:
before the passing the laser fiber mounting assembly, adjusting an axial position of the laser fiber within the laser fiber mounting assembly such that when the laser fiber mounting assembly is coupled with the guide, there is the specified spacing between the tip of the laser fiber and a distal end of the at least one prong.

19. A laser lithotripsy guide system for guiding a laser fiber during a lithotripsy procedure, the guide system comprising:
a mounting assembly securable to a laser fiber; and
a guide comprising:
a body configured to receive the mounting assembly at least partially therein to releasably secure the mounting assembly and the laser fiber to the guide, the body defining a guide bore to receive the laser fiber therethrough when the mounting assembly is secured to the guide; and
at least one prong extending distally from the body, the at least one prong configured to at least partially surround the laser fiber when the laser fiber extends through the guide bore.

20. The guide system of claim 19, wherein the mounting assembly includes:
a collet chuck defining a collet chuck bore to receive the laser through the collet chuck; and
a lock nut defining a lock nut bore to receive the laser fiber through the lock nut, the lock nut threadably securable to the collet chuck to cause the collet chuck to clamp the laser fiber.
